(19) United States
(12) Patent Application Publication
(10) Pub. No.: US 2016/0374797 A1
(43) Pub. Date: Dec. 29, 2016

(54) Breast Implant Delivery System and Method

(71) Applicant: Phi Nguyen, Houston, TX (US)

(72) Inventor: Phi Nguyen, Houston, TX (US)

(21) Appl. No.: 14/751,278

(22) Filed: Jun. 26, 2015

(51) Int. Cl. A61F 2/12

(52) U.S. Cl. CPC A61F 2/12

(57) ABSTRACT

A method of breast augmentation uses a sleeve to insert a sterile silicone breast implant sealed within the sleeve into a surgical pocket in a breast of a human patient, the sleeve having a removable end and wherein the sleeve is transparent or semi-transparent. The method includes cutting the end and grasping the silicone breast implant through the sleeve; and applying manual pressure through the sleeve to the silicone breast implant to push the silicone breast implant toward and through the second opening, without direct hand manipulation of the silicone breast implant, until the silicone breast implant is deposited from the end into the surgical pocket.

This invention is directed to a delivery apparatus for facilitating the insertion of a silicone implant, such as a breast prosthesis, into a surgically developed pocket.

### BACKGROUND OF THE INVENTION

Silicon implant is used for breast augmentation. One problem with silicone implants is that the implants are provided in a filled condition and must be inserted into a surgical pocket. One solution disclosed in U.S. Pat. No. 8,555,893 is a tapered sleeve that includes a lubricating coating on an inner surface. An implant (e.g., a pre-filled silicon breast implant) is introduced into a large end of the sleeve and extruded into a surgical pocket of minimal access incision size through a small-sized end of the apparatus.

### SUMMARY

In one aspect, a method of inserting a silicon breast implant into a surgical pocket of a subject includes lubricating an exterior surface of the silicone breast implant; placing the lubricated silicone breast implant into a sleeve; providing a sealed but openable end in the sleeve; sealing the silicone breast implant in the sleeve for shipment; before surgery, removing the openable end to expose the silicone breast implant; after pre-lubricating the sleeve, grasping the silicone breast implant through the sleeve; and applying manual pressure through the sleeve to the silicone breast implant to push the silicone breast implant toward and through the opening, without direct hand manipulation of the silicone breast implant, until the silicone breast implant is deposited from the second opening into the surgical pocket.

In another aspect, a method is disclosed for using a sealed sleeve to insert a silicone breast implant located within the sleeve into a surgical pocket in a breast of a human patient, the sleeve having an opening. The method includes accessing the implant immediately before surgery by opening the end; grasping the silicone breast implant through the sleeve; and applying manual pressure through the sleeve to the silicone breast implant to push the silicone breast implant toward and through the second opening, without direct hand manipulation of the silicone breast implant, until the silicone breast implant is deposited from the second opening into the surgical pocket.

In a further aspect, a method uses a sleeve to insert a sterile silicone breast implant sealed within the sleeve into a surgical pocket in a breast of a human patient, the sleeve having an openable end. The method includes, prior to surgery, cutting the openable end and grasping the silicone breast implant through the sleeve; and applying manual pressure through the sleeve to the silicone breast implant to push the silicone breast implant toward and through the second opening, without direct hand manipulation of the silicone breast implant, until approximately half of the silicone breast implant has passed through the end, such that the remainder of the silicone breast implant flows out of the end and is deposited into the surgical pocket without additional manual pressure.

In yet another aspect, a method of breast augmentation uses a sleeve to insert a sterile silicone breast implant sealed within the sleeve into a surgical pocket in a breast of a human patient, the sleeve having a removable end and wherein the sleeve is transparent or semi-transparent. The method includes cutting the end and grasping the silicone breast implant through the sleeve; and applying manual pressure through the sleeve to the silicone breast implant to push the silicone breast implant toward and through the second opening, without direct hand manipulation of the silicone breast implant, until the silicone breast implant is deposited from the end into the surgical pocket.

Advantages of the system may include one or more of the following. The process for facilitating the delivery of a silicone implant into a surgically developed pocket. The process allows insertion of a silicone implant through a sleeve defining a small diameter outlet into a patient without direct hand manipulation of the implant. This allows for a “touchless” insertion of a silicone implant into a surgical pocket without any subsequent contamination of the implant. Silicon implants can be inserted through small incisions similar to that achieved by saline implants. The system minimizes potential damage to the outer surface of the implant from excessive pressure applied by hand manipulation of the implant and/or damage associated with a “nick” of the implant surface by a “touching” injury such as a fingernail or insertion that damages the implant. The implantation process is quick and convenient. These and other features, aspects, and advantages of the present invention will become better understood with reference to the following description and appended claims.

### BRIEF DESCRIPTION OF THE DRAWINGS

A fully enabling disclosure of the present invention, including the best mode thereof to one of ordinary skill in the art, is set forth more particularly in the remainder of the specification, including reference to the accompanying drawings.

FIG. 1 is a perspective view of a prosthetic insertion sleeve along with a silicone prosthesis embedded inside to avoid contamination.

FIG. 2 is a view showing manipulation of the prosthesis through the sleeve and illustrating a portion of the prosthesis exiting a tapered end of the sleeve after cutting the tapered end at 24.

### DESCRIPTION OF THE PREFERRED EMBODIMENT

Reference will now be made in detail to the embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used on another embodiment to yield a still. further embodiment. Thus, it is intended that the present invention cover such modifications and variations as come within the scope of the appended claims and their equivalents. Other objects, features, and aspects of the present invention are disclosed in the following detailed description. It is to be understood by one of ordinary skill in the art that the present discussion is a description of exemplary embodiments only and is not intended as limiting the broader aspects of the present invention, which broader aspects are embodied in the exemplary constructions.

In describing the various figures herein, the same reference numbers are used throughout to describe the same material, apparatus, or process pathway. To avoid redundancy, detailed descriptions of much of the apparatus once described in relation to a figure is not repeated in the descriptions of subsequent figures, although such apparatus or process is labeled with the same reference numbers.

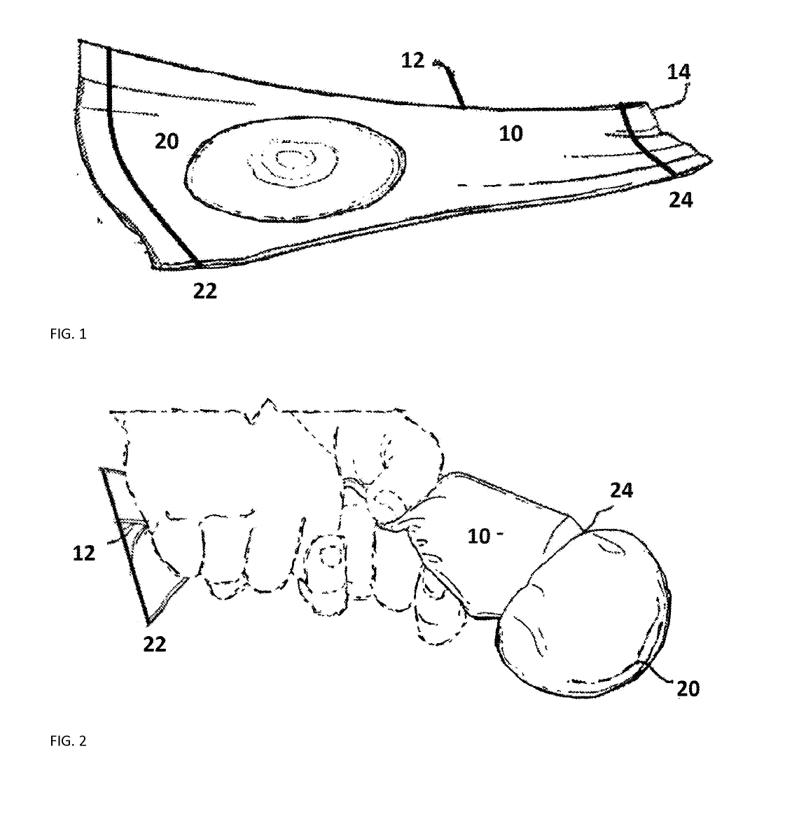

FIG. 1 is a perspective view of a prosthetic insertion sleeve along with a silicone prosthesis embedded inside to avoid contamination while FIG. 2 is a view showing manipulation of the prosthesis through the sleeve and illustrating a portion of the prosthesis exiting a tapered end of the sleeve after cutting the tapered end at 24. As seen in FIG. 1, the implant 20 is sealed inside the sleeve through seals 22 and 24 to protect the patient from organisms that can be carried and shed by the surgeon. These organisms reside on the surgeon's skin, hair, in the nose or mouth, or may be carried on dust particles from the floor or room surfaces. This route of contamination is minor compared to the patient's own flora, however, it is a significant source of contamination is some types of surgery such as orthopedic and central nervous system procedures. The sleeve is additionally sterilized. Sterile gloves are used for handling the sleeve and other surgical procedures. For rodent and non-mammal surgeries (other than aquatics) gloves may be disinfected between surgeries with a cold sterilant.

The surgeon's hands and arms should be scrubbed for 3 minutes with a disinfectant such as povidone iodine or chlorhexidine, rinsed with water and dried prior to gloving for any large animal survival surgery. As much as 30% of the time gloves become perforated during surgery, exposing the animal's tissues directly to the surgeon's skin. If that happens, the surgeon should reglove with sterile gloves. A cap/hair cover, face mask, shoe covers and sterile gown must be worn for surgeries.

As seen in reference to FIGS. 1-2, a sleeve 10 is provided having a general conical shape and which defines a first opening 12 at a larger end of the sleeve and a smaller opening 24 along the tapered terminal tip of sleeve 10. As best seen in reference to FIG. 1, sleeve 10 may be provided by a material which is sufficiently flexible and which enables the sleeve 10 to assume a flattened configuration for storage and shipping. During manufacturing the implant 20, such as a silicone implant, is placed into the interior of sleeve 10, sleeve 10 can be manipulated to conform to the shape of the implant 20 as well as to apply pressure to direct the implant 20 along the length of the sleeve and toward opening 24. The sleeve 10 is then sealed at sealing lines 20 and 22 having the end 24. Prior to insertion into the patient, the seal 22 is cut to allow the silicone implant to be placed into the patient.

Preferably, sleeve 10 is of a flexible material. In one embodiment of the invention, sleeve 10 can be provided by a fabric material such as a plastic-containing fabric which is pliable yet resistant to stretching. It is also envisioned that use of a transparent plastic or other suitable polymer material which has sufficient properties including flexibility and non-elasticity may be used. It is believed that there are advantages to using a transparent or semi-transparent material to assist the surgeon in proper orientation of the implant 20 within the interior of sleeve 10. Suitable transparent materials may include Mylar®, plastics made from Tygon®, brand of plastics, vinyls, polyvinyl chloride, and other similar materials. One suitable material which is flexible and transparent includes compositions of ethylene and alpha-olefin copolymers such as the compositions used in IV saline bags. Suitable multilayer films and sealed structures are taught in U.S. Pat. Nos. 6,743,523 and 7,267,885 both assigned to Baxter International Inc., and which are incorporated herein by reference. As disclosed therein, suitable films heat sealed to form suitable containers, are transparent with minimal hazing, and can be sterilized using gas sterilization or heat with intact seals and remain sufficiently flexible and pliable for the necessary manipulation described herein.

Other attributes of sleeve 10 include the ability to provide sleeve 10 as a sterile component. Accordingly, the material must be capable of withstanding at least one of several conventional sterilization techniques such as a steam autoclave, chemical gas sterilization, or irradiation. Additionally, the interior surface of sleeve 10 may preferably have a low coefficient of friction to facilitate passage of the implant 20 through the sleeve 10. It has been found that using a surgically appropriate lubricant will facilitate passage of the implant 20 through the interior of sleeve 10. Such lubricants may be applied directly to the implant 20 or the sleeve can be coated with lubricant or supplied pre-coated with a lubricant that is already present within sleeve 10.

Since the size of silicone implants 20 may vary in a range from about 150 cc to about 800 cc, the dimensions of opening 24 may be varied to accommodate various size implants 20. Preferably, sleeve 10 is provided with an opening 24 sized to fit the smallest implant. The tip opening 24 may be enlarged by cutting portions of the sleeve to provide a larger opening. If desired, indicia may be present on the exterior of sleeve 10 to allow the cutting of the sleeve to the proper dimensions for the size of the implant 20.

Alternatively, the tip opening 24 can have a diameter sized to allow the largest standard implant of 800 cc to exit the sleeve. According to this invention, it has been found that an opening 24 having a diameter of about 6 cm will allow delivery of the implant into the surgical pocket. As described below, use of an optional tip would allow for a sleeve to be provided having a single opening size to accommodate the largest implant. Selection of an appropriate tip can then be made based upon the size of the implant.

While it is believed preferable that sleeve 10 be provided from a single structural substrate, it is also recognized that an equivalent device can be provided of a flexible sleeve 10 having a separate inner liner (not illustrated) which may be present within the interior of sleeve 10. The liner could either be integral with sleeve 10 or may be a separate layer of material manually inserted within the interior of sleeve 10 at the time of use.

The larger opening 12 of sleeve 10 allows the implant to be placed within the sleeve with little force or manual manipulation. When the implant is within the interior of sleeve 10, the larger opening may be twisted closed as seen in FIGS. 2 and 3. Thereafter, the surgeon is able to apply manual pressure via the sleeve to the implant 20. The surgeon is thus able to apply pressure to the implant, forcing the implant toward the smaller opening 24. As seen in reference to FIGS. 2 and 3, the implant can be forced through the small opening 24.

As the surgeon is manipulating the implant through sleeve 10, the opening 22 is placed within the surgical pocket designed for receiving the implant. Accordingly, tip 24 is inserted through an incision associated with the surgical pocket. As the implant is forced through opening 24, the surgical pocket can be manipulated slightly to create a vacuum that assists in the placement of the implant into the pocket. Additionally, another useful feature of the apparatus and process is that as approximately half of the implant 20 has been exerted through opening 24, the remainder of the implant will flow through the sleeve without additional manipulation. Accordingly, once the opening 24 is positioned within the surgical pocket, implant 20 can be manipulated so that the prosthesis 20 is forced into the surgical pocket. The surgeon is able to control the positioning and orientation of the implant 20 by proper rotation and positioning of the sleeve 10 containing the implant 20.

It has been found that use of a sleeve 10 can greatly reduce the amount of time required for insertion of an implant 20. It has been found that the step of inserting a simple implant can occur within a timeframe of about 3 to 20 seconds minutes as compared to a time interval of 5 to 15 minutes for a traditional hand manipulation of an implant. Additionally, because the implant can be inserted through a small opening, the size of the surgical incision can be made smaller than would otherwise be required for a silicone implant.

The use of the sleeve 10 and implant 20 can be used with incisions. For instance, periareolar, trans axillary, intramammary incisions can be used with the above process and apparatus for insertion of an implant.

In accordance with this invention, it has been found beneficial to initially lubricate the exterior of implant 20 with an appropriate surgical lubricant such as K-Y® brand sterile lubricant. Following lubrication, the lubricated implant 20 is placed within the sleeve 10 and the implant 20 is forced through opening 24 as a pre-lubrication step. Following this pre-lubrication step, the implant can again be placed within the sleeve and subsequently inserted into the patient's surgical pocket.

It is also envisioned that, depending upon the coefficient of friction of the interior of sleeve 10 and/or any associated liner, it may be possible to provide other types of lubricants, including dry or powdered lubricant products to the interior of sleeve 10. Such lubricants are activated by being moistened and would provide an alternative to manually coating the prosthesis with a lubricant.

If desired, sleeve 10 can further define a structural tip (not illustrated) in association with opening 24. The tip could be provided of a more elastic material that facilitates insertion of the tip into the surgical pocket. For instance, a separate tip could provide for an extension beyond the existing opening 24 and which would have a narrower initial diameter providing a longer tip which may be more easily inserted within the interior of a surgical pocket. The ability of an optional tip to expand allows the implant 20 to pass through sleeve 10 and opening 24 while positioning the exiting implant 20 further within the surgical pocket. The use of the tip is believed beneficial in that it prevents the passage of the implant from extruding the sleeve from the incision. In other words, the tip provides a deeper positioning for the sleeve 10 more accurately directs the placement of the implant within the surgical pocket. The use of an expandable tip may facilitate the insertion time and lessen the learning curve for surgeons who are using the sleeve 10.

An important attribute of sleeve 10, including any optional tip structure 24, is that the interior surface of sleeve 10, including opening 24 and any associated tip member, must provide for a smooth and substantially uninterrupted passageway. It is important that any seams that may be formed or abutments between one type of material to another or from opening 24 to an associated tip must be of a sufficient smoothness such that the surface of the implant 20 is not degraded. Accordingly, it is envisioned that sonically welded seams or the use of a unitary extrusion process is desired for forming appropriate sleeves 10. In addition, to the extent opening 24 may be “cut to size”, it is important that the material, once cut, not present any cutting artifacts or roughened edges that could damage the implant 20. Similarly, the identical concerns must be met by any optional tip used with sleeve 10 such that the material of the tip as well as its method of attaching the tip to the sleeve 10 and sleeve opening 24 must not present any potential implant contact surfaces that could result in damage to the implant.

Although preferred embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present invention which is set forth in the following claims. In addition, it should be understood that aspects of the various embodiments may be interchanged, both in whole, or in part. Therefore, the spirit and scope of the appended claims should not be limited to the description of the preferred versions contained therein.

What is claimed is:

1. A method of inserting a silicon breast implant into a surgical pocket of a subject, comprising:
lubricating an exterior surface of the silicone breast implant;
placing the lubricated silicone breast implant into a sleeve;
providing a sealed but openable end in the sleeve;
sealing the silicone breast implant in the sleeve for shipment;
before surgery, removing the openable end to expose the silicone breast implant;
after pre-lubricating the sleeve, grasping the silicone breast implant through the sleeve; and applying manual pressure through the sleeve to the silicone breast implant to push the silicone breast implant toward and through the opening, without direct hand manipulation of the silicone breast implant, until the silicone breast implant is deposited from the second opening into the surgical pocket.

2. A method of using a sealed sleeve to insert a silicone breast implant located within the sleeve into a surgical pocket in a breast of a human patient, the sleeve having an opening, the method comprising: accessing the implant immediately before surgery by opening the end; grasping the silicone breast implant through the sleeve; and applying manual pressure through the sleeve to the silicone breast implant to push the silicone breast implant toward and through the second opening, without direct hand manipulation of the silicone breast implant, until the silicone breast implant is deposited from the second opening into the surgical pocket.

3. The method of claim 2, wherein the sleeve is transparent or semi-transparent.

4. The method of claim 3, wherein the sleeve is tapered.

5. The method of claim 2, wherein the sleeve comprises a liner.

6. The method of claim 2, wherein the sleeve is generally conical.

7. The method of claim 2, wherein the sleeve is pre-coated with a lubricant.

8. A method of using a sleeve to insert a silicone breast implant sealed within the sleeve into a surgical pocket in a breast of a human patient, the sleeve having ara openable end, the method comprising:
prior to surgery, cutting the openable end and grasping the silicone breast implant through the sleeve; and
applying manual pressure through the sleeve to the silicone breast implant to push the silicone breast implant toward and through the second opening, without direct hand manipulation of the silicone breast implant, until approximately half of the silicone breast implant has passed through the end, such that the remainder of the silicone breast implant flows out of the end and is deposited into the surgical pocket without additional manual pressure.

9. The method of claim 8, wherein the sleeve is transparent or semi-transparent.

10. The method of claim 9, wherein the sleeve is tapered.

11. The method of claim 8, wherein the sleeve comprises a liner.

12. The method of claim 8, wherein the sleeve is generally conical.

13. The method of claim 8, wherein the sleeve is pre-coated with a lubricant.

24. A method of using a sleeve to insert a silicone breast implant sealed within the sleeve into a surgical pocket in a breast of a human patient, the sleeve having a removable end and wherein the sleeve is transparent or semi-transparent, the method comprising:
cutting the end and grasping the silicone breast implant through the sleeve; and
applying manual pressure through the sleeve to the silicone breast implant to push the silicone breast implant toward and through the second opening, without direct hand manipulation of the silicone breast implant, until the silicone breast implant is deposited from the end into the surgical pocket.

15. The method of claim 24, further comprising observing the silicone breast implant through the sleeve to control the position and orientation of the silicone breast implant by proper rotation and positioning of the sleeve.

16. The method of claim 15, wherein the sleeve is tapered.

17. The method of claim 24, wherein the sleeve comprises a liner.

18. The method of claim 24, wherein the sleeve is generally conical.

19. The method of claim 24, wherein the sleeve is pre-coated with a lubricant.

20. The method of claim 24, comprising cutting the tapered end prior to inserting the implant into a patient.
